(19) United States
(12) Patent Application Publication
(10) Pub. No.: US 2024/0366634 A1
(43) Pub. Date: Nov. 7, 2024

(54) METHODS FOR THE TREATMENT OF CB1-, TRPA1- AND TRPV1-DEPENDENT CONDITIONS

(71) Applicant: BUZZELET DEVELOPMENT AND TECHNOLOGIES LTD., Or Akiva (IL)

(72) Inventors: Noa RAZ, Gizo (IL); Aharon EYAL, Jerusalem (IL)

(21) Appl. No.: 18/688,778

(22) PCT Filed: Sep. 2, 2022
(86) PCT No.: PCT/IB2022/058245

§ 371 Date: Mar. 3, 2024

Related U.S. Application Data
(60) Provisional application No. 63/240,379, filed on Sep. 3, 2021.
(60) Provisional application No. 63/337,111, filed on May 1, 2022.
(60) Provisional application No. 63/351,389, filed on Jun. 12, 2022.

(51) Int. Cl. A61K 31/00

(52) U.S. Cl. CPC A61K 31/658

(57) ABSTRACT

Provided is a method for treating a condition related to cannabinoid type 1 (CB1) receptor activation by administering a composition comprising at least one terpene, optionally together with tetrahydrocannabinol (THC). Further provided is a method for treating a condition related to Transient Receptor Potential Cation Channel, subfamily A, member 1 (TRPA1) receptor activation by administering a composition comprising at least one terpene selected from the group consisting of linalool, menthol, eucalyptol, terpincol, citral and combinations thereof, optionally together with tetrahydrocannabinol (THC). Further provided is a method for treating a condition related to Transient Receptor Potential Cation Channel, subfamily V, member 1 (TRPA1) receptor activation by administering a composition comprising at least one terpene selected from the group consisting of citral, caryophyllene and combinations thereof, optionally together with cannabidiol (CBD).

### CROSS-REFERENCE TO RELATED APPLICATIONS

The present application gains priority from U.S. Provisional Patent Application Ser. No. 63/240,379 filed Sep. 3, 2021; U.S. Provisional Patent Application Ser. No. 63/337,111 filed May 1, 2022; and U.S. Provisional Patent Application Ser. No. 63/351,389 filed Jun. 12, 2022, all of which are incorporated by reference as if fully set-forth herein.

### FIELD OF THE INVENTION

The present invention relates to the field of medical treatment, and more specifically to methods for the treatment of conditions related to at least one of CB1, TRPA1 and TRPV1 receptor activity, using terpenes for modulation of such receptor activity.

### BACKGROUND OF THE INVENTION

Cannabinoid receptor 1 (CB1) is a G protein coupled cannabinoid receptor, that is expressed in humans in the peripheral and central nervous systems, and is activated by endocannabinoids, plant phytocannabinoids and synthetic analogs of THC. Activation and deactivation of the activity of a CB1 receptor affects a wide range of physiological functions and conditions in the body of a subject, such as a human subject.

Transient receptor potential cation channel subfamily A, member 1 (TRPA1) is a protein located on the plasma membrane of many human and animal cells, which serves as a sensor for sensations such as pain, cold and itching in humans and other mammals.

Transient receptor potential cation channel subfamily V, member 1 (TRPV1) is a non-selective cation channel that may be activated by a wide variety of exogenous and endogenous physical and chemical stimuli and plays a role in modulation of body temperature.

Each of the above receptors provides a potential therapeutic target for treatment of various adverse conditions associated with the receptor.

### SUMMARY OF THE INVENTION

According to an aspect of some embodiments of the present invention, there is provided a method for treating a condition related to cannabinoid type 1 (CB1) receptor activation or a symptom thereof in a subject in need thereof, the method comprising administering to the subject a composition comprising at least one terpene.

According to a further aspect of some embodiments of the present invention, there is provided a composition comprising at least one terpene for use in treating a condition related to cannabinoid type 1 (CB1) receptor activation or a symptom thereof by modulating CB1-receptor activation.

According to a further aspect of some embodiments of the present invention, there is provided a method for treating a condition related to Transient Receptor Potential Cation Channel, subfamily A, member 1 (TRPA1) receptor activation in a subject in need thereof, the method comprising modulating TRPA1 receptor activation by administering to the subject a composition comprising at least one terpene selected from the group consisting of linalool, menthol, eucalyptol, terpineol, citral and combinations thereof.

According to a further aspect of some embodiments of the present invention, there is provided a composition comprising at least one terpene selected from the group consisting of linalool, menthol, eucalyptol, terpineol, citral and combinations thereof for use in treating a condition related to Transient Receptor Potential Cation Channel, subfamily A, member 1 (TRPA1) receptor activation by modulating TRPA1 receptor activation.

According to a further aspect of some embodiments of the present invention, there is provided a method for treating a condition related to Transient Receptor Potential Cation Channel, subfamily V, member 1 (TRPV1) receptor activation in a subject in need thereof, the method comprising modulating TRPV1 activation by administering to the subject a composition comprising at least one terpene selected from the group consisting of citral, caryophyllene and combinations thereof.

According to a further aspect of some embodiments of the present invention, there is provided a composition comprising at least one terpene selected from the group consisting of linalool, menthol, eucalyptol, terpineol, citral and combinations thereof for use in treating a condition related to Transient Receptor Potential Cation Channel, subfamily V, member 1 (TRPV1) receptor activation by modulating TRPV1 receptor activation.

### DETAILED DESCRIPTION OF THE INVENTION

The present invention relates to methods for the treatment of conditions dependent on at least one of CB1 receptor activation, TRPA1 receptor activation and TRPV1 receptor activation.

Unless otherwise defined, all technical and scientific terms used herein have the same meaning as commonly understood by one of ordinary skill in the art to which this invention belongs.

The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the terms “conditions related to CB1 receptor activation”, “conditions related to TRPA1 receptor activation” and “conditions related to TRPV1 receptor activation” refer to conditions caused by modulation of CB1, TRPA1 or TRPV1 receptor activity, respectively. Such activity may comprise increasing or decreasing activation of the receptors, such as by binding to the receptors, increasing or decreasing binding of an agonist to the receptors and/or activation of the receptors, increasing or decreasing deactivation of receptors by an antagonist

The terms “conditions related to CB1 receptor activation”, “conditions related to TRPA1 receptor activation” and “conditions related to TRPV1 receptor activation” as used herein, also refer to conditions and/or symptoms associated with the activation or inhibition of the receptors. “conditions related to CB1 receptor activation” include the sensation of pain, appetite, sleep, mood, memory and anxiety. “conditions related to TRPA1 receptor activation” include the sensation of pain, cold and itch. “conditions related to TRPV1 receptor activation” include the sensation of pain and heat.

As used herein, the term “modulating” with regard to activation of a receptor refers to changing, controlling, affecting, managing or influencing a type or magnitude of activation of the receptor, and includes increasing or decreasing a magnitude, a frequency and/or a severity of the receptor activation. “Modulating of activation of a receptor” may be applied to an abnormal physiological function for treating a condition or a symptom thereof arising from such abnormal function, or to a normal healthy physiological function in order to provide a desired change in the function. Modulation of activation of the receptor may be achieved, for example by increasing or decreasing activity of the receptor; increasing or decreasing availability and/or expression of the receptor; or by increasing or decreasing availability of an agonist to the receptor. Alternatively, modulation may be achieved by increasing or decreasing binding of the agonist to the receptor.

As used herein, the term “treating” includes ameliorating, mitigating, and reducing the instances of a disease or condition, or the symptoms of a disease or condition.

As used herein, the term “administering” includes any mode of administration, such as oral, subcutaneous, sublingual, transmucosal, parenteral, intravenous, intra-arterial, buccal, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

As used herein, the term “therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

Reference to a specified terpene, as used herein, is intended to encompass molecules having a similar molecular structure to the specified terpene. As used herein, the term “molecules having a similar molecular structure” to a specified terpene refers to molecules having the same number of carbon atoms as the specified terpene and further having at least two, such as two, three or four of the following properties:
a) Differing from the specified terpene by no more than one oxygen atoms, e.g. when the specified terpene has zero oxygen atoms, a molecule having a similar molecular structure may have one oxygen atom; and when the specified terpene has one oxygen atom, a molecule having a similar structure may have zero or two oxygen atoms.
b) Differing from the specified terpene by no more than one double bond, e.g. when the specified terpene has one double bond, a molecule having a similar molecular structure may have two double bonds; when the specified terpene has two double bonds, a molecule having a similar structure may have one or three double bonds; and when the specified terpene has three double bonds, a molecule having a similar molecular structure may have two double bonds.
c) Differing from the specified terpene by no more than one ring structure, e.g. when the specified terpene is devoid of a ring structure (i.e. is linear), a molecule having a similar molecular structure may have one ring structure; when the specified terpene has one ring structure, a molecule having a similar structure may have zero or two ring structures; and when the specified terpene has two ring structures, a molecule having a similar molecular structure may have one ring structure.
d) When the specified terpene includes at least one double bond, a molecule having a similar molecular structure has a same isomer (cis or trans) on at least one double bond.
e) When the specified terpene includes at least one chiral center, a molecule having a similar molecular structure has the same chirality on at least one chiral center.s

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”. As used herein, the terms “water content” and “moisture content” are used interchangeably.

Unless indicated otherwise, percent is weight percent and ratio is weight/weight ratio.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a method for treating a condition related to cannabinoid type 1 (CB1) receptor activation or a symptom thereof in a subject in need thereof, the method comprising modulating CB1-receptor activation by administering to the subject a composition comprising at least one terpene.

According to some embodiments, treating further comprises administering to the subject tetrahydrocannabinol (THC) at a total terpene to THC weight/weight ratio in a range between about 0.05:1 and about 1:1 (such as about 0.05:1, about 0.06:1, about 0.07:1, about 0.08:1, about 0.09:1, about 0.1:1, about 0.2:1, about 0.3:1, about 0.4:1, about 0.5:1, about 0.6:1, about 0.7:1, about 0.8:1, about 0.9:1 or about 1:1), wherein modulating CB1-receptor activation is via modulating an interaction of THC with the CB1 receptor.

According to some embodiments, THC is administered to the subject at a dosage of from about 1 to about 100 mg (such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg) THC.

According to some embodiments, THC is administered to the subject at a dosage resulting in a concentration of up to about 10 μM (such as about 1 μM, about 2 μM, about 3 μM, about 4 μM, about 5 μM, about 6 μM, about 7 μM, about 8 μM, about 9 μM, or about 10 μM) of a total concentration of the at least one terpene in a plasma and/or serum of the subject.

According to some embodiments, the at least one terpene (such as one terpene, two terpenes, three terpenes, four terpenes, five terpenes, six terpenes or seven terpenes) is selected from the group consisting of linalool, limonene, geraniol, ocimene, borneol, terpineol, sabinene and combinations thereof.

According to some embodiments, the at least one terpene comprises at least three terpenes (such as three terpenes, four terpenes, five terpenes, six terpenes, seven terpenes or eight terpenes) selected from the group consisting of alpha pinene, myrcene, limonene, linalool, caryophyllene, humulene, nerolidol and bisabolol. According to some such embodiments, alpha pinene, when present, is at a concentration of from about 5 to about 20 wt % of the total terpene content; myrcene, when present, is at a concentration of from about 5 to about 25 wt % of the total terpene content; limonene, when present, is at a concentration of from about 1 to about 15 wt % of the total terpene content; linalool, when present is at a concentration of from about 3 to about 20 wt % of the total terpene content; caryophyllene, when present, is at a concentration of from about 15 to about 35 wt % of the total terpene content; humulene, when present, is at a concentration of from about 1 to about 15% of the total terpene content; nerolidol, when present, is at a concentration of from about 5 to about 25 wt % of the total terpene content; and bisabolol, when present, is at a concentration of from about 5 to about 20 wt % of the total terpene content.

According to some embodiments, the at least one terpene comprises at least three terpenes (such as three terpenes, four terpenes or five terpenes) selected from the group consisting of alpha pinene, limonene, terpinolenes, caryophyllene, and bisabolol. According to some such embodiments, alpha pinene, when present, is at a concentration of from about 15 to about 35 wt % of the total terpene content; limonene, when present, is at a concentration of from about 10 to about 30 wt % of the total terpene content; terpinolene, when present, is at a concentration of from about 5 to about 25 wt % of the total terpene content; caryophyllene, when present, is at a concentration of from about 15 to about 35 wt % of the total terpene content; and bisabolol, when present, is at a concentration of from about 3 to about 20 wt % of the total terpene content.

According to some embodiments, the at least one terpene comprises at least three terpenes (such as three terpenes, four terpenes, five terpenes, six terpenes, seven terpenes, eight terpenes or nine terpenes) selected from the group consisting of beta pinene, myrcene, ocimene, linalool, terpineol, borneol, caryophyllne, nerolidol and bisabolol. According to some such embodiments, beta pinene, when present, is at a concentration of from about 5 to about 25 wt % of the total terpene content; myrcene, when present, is at a concentration of from about 3 to about 25 wt % of the total terpene content; ocimene, when present, is at a concentration of from about 3 to about 20 wt % of the total terpene content; linalool, when present, is at a concentration of from about 5 to about 25 wt % of the total terpene content; terpineol, when present, is at a concentration of from about 5 to about 25 wt % of the total terpene content; borneol, when present, is at a concentration of from about 5 to about 25 wt % of the total terpene content; caryophyllene, when present, is at a concentration of from about 10 to about 30 wt % of the total terpene content; nerolidol, when present, is at a concentration of from about 5 to about 25 wt % of the total terpene content; and bisabolol, when present, is at a concentration of from about 3 to about 20 wt % of the total terpene content

According to some embodiments, the least one terpene comprises at least three terpenes (such as three terpenes, four terpenes, five terpenes, six terpenes or seven terpenes) selected from the group consisting of alpha pinene, sabinene, limonene, terpinolene, eucalyptol, borneol and caryophyllene. According to some such embodiments, alpha pinene, when present, is at a concentration of from about 5 to about 25 wt % of the total terpene content; sabinene, when present, is at a concentration of from about 5 to about 25 wt % of the total terpene content; limonene, when present, is at a concentration of from about 5 to about 25 wt % of the total terpene content; w terpinolene, when present, is at a concentration of from about 1 to about 15 wt % of the total terpene content; eucalyptol, when present, is at a concentration of from about 5 to about 25 wt % of the total terpene content; borneol, when present, is at a concentration of from about 5 to about 25 wt % of the total terpene content; and caryophyllene, when present is at a concentration of from about 5 to about 30 wt % of the total terpene content.

According to some embodiments, the at least one terpene comprises at least three terpenes (such as three terpenes, four terpenes, five terpenes, six terpenes, seven terpenes or eight terpenes) selected from the group consisting of alpha pinene, beta pinene, eucalyptol, linalool, terpineol, borneol, caryophyllene and nerolidol. According to some such embodiments, alpha pinene, when present, is at a concentration of from about 5 to about 25 wt % of the total terpene content; beta pinene, when present, is at a concentration of from about 1 to about 15 wt % of the total terpene content; eucalyptol, when present is at a concentration of from about 3 to about 25 wt % of the total terpene content; linalool, when present, is at a concentration of from about 3 to about 25 wt % of the total terpene content; terpineol, when present, is at a concentration of from about 3 to about 25 wt % of the total terpene content; borneol, when present, is at a concentration of from about 3 to about 25 wt % of the total terpene content; caryophyllene, when present, is at a concentration of from about 5 to about 25 wt % of the total terpene content; and nerolidol, when present is at a concentration of from about 3 to about 25 wt % of the total terpene content.

According to some embodiments, the at least one terpene comprises at least three terpenes (such as three terpenes, four terpenes or five terpenes) selected from the group consisting of alpha pinene, myrcene, caryophyllene, nerolidol and bisabolol. According to some such embodiments, alpha pinene, when present, is at a concentration of from about 5 to about 25 wt % of the total terpene content; myrcene, when present, is at a concentration of from about 15 to about 35 wt % of the total terpene content; caryophyllene, when present, is at a concentration of from about 15 to about 35 wt % of the total terpene content; nerolidol, when present is at a concentration of from about 15 to about 35 wt % of the total terpene content; and bisabolol, when present, is at a concentration of from about 3 to about 25 wt % of the total terpene content.

According to some embodiments, the at least one terpene comprises at least two terpenes (such as two terpenes, three terpenes, four terpenes or five terpene) selected from the group consisting of limonene, alpha pinene, borneol, bisabolol and myrcene.

According to some embodiments, the at least one terpene (such as one terpene, two terpenes, three terpenes, five terpenes, six terpenes, seven terpenes, eight terpenes or nine terpenes) is selected from the group consisting of alpha pinene, beta pinene, limonene, terpineol, geraniol, myrcene, ocimene, terpinolene, eucalyptol and combinations thereof.

According to some embodiments, the at least one terpene (such as one terpene, two terpenes, three terpenes, five terpenes, six terpenes, seven terpenes, eight terpenes, nine terpenes, ten terpenes, eleven terpenes, twelve terpenes or thirteen terpenes) is selected from the group consisting of alpha-pinene, beta-pinene, ocimene, limonene, linalool, geraniol, caryophyllene, humulene, terpinolene, nerolidol, valencene, bulnesene, selina diene and combinations thereof.

According to some embodiments, the at least one terpene comprises one terpene, two terpenes, three terpenes or all four terpenes selected from the group consisting of beta-pinene, ocimene, limonene, linalool and combinations thereof. According to some such embodiments, the method further comprises administering THC, wherein a total weight of the at least one terpene in the composition is about 0.05-0.3 (such as about 0.05, about 0.1, about 0.15, about 0.2, about 0.25 or about 0.3) of a weight of the THC.

According to some embodiments, the at least one terpene comprises at least three terpenes (such as three terpenes, four terpenes, five terpenes, or all six terpenes) selected from the group consisting of alpha-pinene, beta-pinene, caryophyllene, linalool, limonene, myrcene and combinations thereof. According to some such embodiments, alpha pinene, when present, is at a concentration of from about 5 to about 25 wt % of the total terpene content; beta pinene, when present, is at a concentration of from about 5 to about 25 wt % of the total terpene content; limonene, when present, is at a concentration of from about 5 to about 25 wt % of the total terpene content; linalool, when present, is at a concentration of from about 5 to about 25 wt % of the total terpene content; myrcene, when present, is at a concentration of from about 5 to about 25 wt % of the total terpene content; and caryophyllene, when present is at a concentration of from about 5 to about 25 wt % of the total terpene content. According to some such embodiments, the method further comprises administering THC, wherein a total weight of the at least one terpene in the composition is about 0.05-0.3 (such as about 0.05, about 0.1, about 0.15, about 0.2, about 0.25 or about 0.3) of a weight of the THC.

According to some embodiments, the at least one terpene comprises at least three terpenes (such as three terpenes, four terpenes, five terpenes, six terpenes or seven terpenes) selected from the group consisting of alpha-pinene, beta-pinene, caryophyllene, linalool, limonene, terpinolene, bisabolol and combinations thereof. According to some such embodiments, the method further comprises administering THC, wherein a total weight of the at least one terpene in the composition is about 0.05-0.3 (such as about 0.05, about 0.1, about 0.15, about 0.2, about 0.25 or about 0.3) of a weight of the THC.

According to some embodiments, the at least one terpene (such as one terpene, two terpenes or three terpenes) is selected from the group consisting of alpha-pinene, humulene, caryophyllene and combinations thereof. According to some such embodiments, the method further comprises administering THC, wherein a total weight of the at least one terpene in the composition is about 0.05-0.3 (such as about 0.05, about 0.1, about 0.15, about 0.2, about 0.25 or about 0.3) of a weight of the THC.

According to some embodiments, the at least one terpene comprises at least three terpenes (such as three terpenes, four terpenes, five terpenes, six terpenes or seven terpenes) selected from the group consisting of alpha-pinene, beta-pinene, limonene, terpinolene, geraniol, caryophyllene, bisabolol and combinations thereof. According to some such embodiments, alpha pinene, when present, is at a concentration of from about 5 to about 25 wt % of the total terpene content; beta pinene, when present, is at a concentration of from about 1 to about 15 wt % of the total terpene content; limonene, when present, is at a concentration of from about 5 to about 25 wt % of the total terpene content; terpinolene, when present, is at a concentration of from about 5 to about 25 wt % of the total terpene content; geraniol, when present is at a concentration of from about 5 to about 25 wt % of the total terpene content; caryophyllene, when present, is at a concentration of from about 10 to about 30 wt % of the total terpene content; and bisabolol, when present, is at a concentration of from about 5 to about 25 wt % of the total terpene content. According to some such embodiments, the method further comprises administering THC, wherein a total weight of the at least one terpene in the composition is about 0.05-0.3 (such as about 0.05, about 0.1, about 0.15, about 0.2, about 0.25 or about 0.3) of a weight of the THC.

According to some embodiments, the composition comprising the at least one terpene is administered in a separate composition from the THC.

According to some embodiments, the composition comprising at least one terpene is administered prior to administration of the THC.

According to some embodiments, the composition comprising at least one terpene is administered subsequent to administration of the THC.

According to some embodiments, the composition comprising at least one terpene is administered within 2 hours of administration of the THC.

According to some embodiments, the composition comprising at least one terpene is in a form selected from the group consisting of a vapor and an aerosol.

According to some embodiments, the THC is administered sublingually.

According to some embodiments, the composition comprising at least one terpene is administered in a liquid form.

According to some embodiments, the modulation of an interaction of the THC with the CB1 receptor is selected from the group consisting of an allosteric modulation, an orthosteric modulation, and a combination thereof.

According to some embodiments, the condition related to cannabinoid type 1 (CB1) receptor-activation is selected from the group consisting of pain, a sleep disorder, an appetite disorder, anxiety, depression, a memory disorder, a movement disorder, inflammation, abnormal excitatory neuronal activity, neurodegeneration, abnormal neurotransmitter release, stress, a cardiovascular function disorder, a motivation disorder, a mood disorder, a sedation disorder, a cognitive function disorder, abnormal muscle tension, cramps and combinations thereof.

According to an aspect of some embodiments of the present invention, there is provided a composition comprising at least one terpene for use in treating a condition related to cannabinoid type 1 (CB1) receptor activation or a symptom thereof by modulating CB1-receptor activation.

According to some embodiments, the composition for use is for administering together with tetrahydrocannabinol (THC) at a total terpene to THC weight/weight ratio in a range between about 0.05:1 and about 1:1 (such as about 0.05:1, about 0.06:1, about 0.07:1, about 0.08:1, about 0.09:1, about 0.1:1, about 0.2:1, about 0.3:1, about 0.4:1, about 0.5:1, about 0.6:1, about 0.7:1, about 0.8:1, about 0.9:1 or about 1:1), wherein modulating CB1-receptor activation is via modulating an interaction of THC with the CB1 receptor.

According to some embodiments, THC is for administering to a subject at a dosage of from about 1 to about 100 mg (such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg. about 80 mg. about 85 mg, about 90 mg. about 95 mg or about 100 mg) THC.

According to some embodiments, THC is for administering to a subject at a dosage resulting in a concentration of up to about 10 μM (such as about 1 μM, about 2 μM, about 3 μM, about 4 μM, about 5 μM, about 6 μM, about 7 μM, about 8 μM, about 9 μM, or about 10 μM) of a total concentration of the at least one terpene in a plasma and/or serum of the subject.

According to some embodiments, the at least one terpene (such as one terpene, two terpenes, three terpenes, four terpenes, five terpenes, six terpenes or seven terpenes) is selected from the group consisting of linalool, limonene, geraniol, ocimene, borneol, terpineol, sabinene and combinations thereof.

According to some embodiments, the at least one terpene comprises at least three terpenes (such as three terpenes, four terpenes, five terpenes, six terpenes, seven terpenes or eight terpenes) selected from the group consisting of alpha pinene, myrcene, limonene, linalool, caryophyllene, humulene, nerolidol and bisabolol.

According to some embodiments, the at least one terpene comprises at least three terpenes (such as three terpenes, four terpenes, five terpenes, six terpenes or seven terpenes) selected from the group consisting of alpha pinene, limonene, linalool, caryophyllene, humulene, nerolidol and bisabolol.

According to some embodiments, the at least one terpene comprises at least three terpenes (such as three terpenes, four terpenes, five terpenes, six terpenes, seven terpenes, eight terpenes or nine terpenes) selected from the group consisting of beta pinene, myrcene, ocimene, linalool, terpineol, borneol, caryophyllne, nerolidol and bisabolol.

According to some embodiments, the least one terpene comprises at least three terpenes (such as three terpenes, four terpenes, five terpenes, six terpenes or seven terpenes) selected from the group consisting of alpha pinene, sabinene, limonene, terpinolene, eucalyptol, borneol and caryophyllene.

According to some embodiments, the at least one terpene comprises at least two terpenes (such as two terpenes, three terpenes, four terpenes or five terpene) selected from the group consisting of limonene, alpha pinene, borneol, bisabolol and myrcene.

According to some embodiments, the at least one terpene (such as one terpene, two terpenes, three terpenes, five terpenes, six terpenes, seven terpenes, eight terpenes or nine terpenes) is selected from the group consisting of alpha pinene, beta pinene, limonene, terpineol, geraniol, myrcene, ocimene, terpinolene, eucalyptol and combinations thereof.

According to some embodiments, the at least one terpene (such as one terpene, two terpenes, three terpenes, five terpenes, six terpenes, seven terpenes, eight terpenes, nine terpenes, ten terpenes, eleven terpenes, twelve terpenes or thirteen terpenes) is selected from the group consisting of alpha-pinene, beta-pinene, ocimene, limonene, linalool, geraniol, caryophyllene, humulene, terpinolene, nerolidol, valencene, bulnesene, selina diene and combinations thereof.

According to some embodiments, the at least one terpene comprises one terpene, two terpenes, three terpenes or all four terpenes selected from the group consisting of beta-pinene, ocimene, limonene, linalool and combinations thereof. According to some such embodiments, the composition is for administering together with THC, wherein a total weight of the at least one terpene in the composition is about 0.05-0.3 (such as about 0.05, about 0.1, about 0.15, about 0.2, about 0.25 or about 0.3) of a weight of the THC.

According to some embodiments, the at least one terpene comprises at least three terpenes (such as three terpenes, four terpenes, five terpenes, or all six terpenes) selected from the group consisting of alpha-pinene, beta-pinene, caryophyllene, linalool, limonene, myrcene and combinations thereof. According to some such embodiments, the composition is for administering together with THC, wherein a total weight of the at least one terpene in the composition is about 0.05-0.3 (such as about 0.05, about 0.1, about 0.15, about 0.2, about 0.25 or about 0.3) of a weight of the THC.

According to some embodiments, the at least one terpene comprises at least three terpenes (such as three terpenes, four terpenes, five terpenes, six terpenes or seven terpenes) selected from the group consisting of alpha-pinene, beta-pinene, caryophyllene, linalool, limonene, terpinolene, bisabolol and combinations thereof. According to some such embodiments, the composition is for administering together with THC, wherein a total weight of the at least one terpene in the composition is about 0.05-0.3 (such as about 0.05, about 0.1, about 0.15, about 0.2, about 0.25 or about 0.3) of a weight of the THC.

According to some embodiments, the at least one terpene (such as one terpene, two terpenes or three terpenes) is selected from the group consisting of alpha-pinene, humulene, caryophyllene and combinations thereof. According to some such embodiments, the composition is for administering together with THC, wherein a total weight of the at least one terpene in the composition is about 0.05-0.3 (such as about 0.05, about 0.1, about 0.15, about 0.2, about 0.25 or about 0.3) of a weight of the THC.

According to some embodiments, the at least one terpene comprises at least three terpenes (such as three terpenes, four terpenes, five terpenes, six terpenes or seven terpenes) selected from the group consisting of alpha-pinene, beta-pinene, limonene, terpinolene, geraniol, caryophyllene, bisabolol and combinations thereof. According to some such embodiments, the composition is for administering together with THC, wherein a total weight of the at least one terpene in the composition is about 0.05-0.3 (such as about 0.05, about 0.1, about 0.15, about 0.2, about 0.25 or about 0.3) of a weight of the THC.

According to some embodiments, the composition comprising the at least one terpene is for administering in a separate composition from the THC.

According to some embodiments, the composition comprising at least one terpene is for administering prior to administration of the THC.

According to some embodiments, the composition comprising at least one terpene is administered subsequent to administration of the THC.

According to some embodiments, the composition comprising at least one terpene is for administering within 2 hours of administration of the THC.

According to some embodiments, the composition comprising at least one terpene is in for administering in a form selected from the group consisting of a vapor and an aerosol.

According to some embodiments, the THC is for administering sublingually.

According to some embodiments, the composition comprising at least one terpene is for administering in a liquid form.

According to some embodiments, the modulation of an interaction of THC with the CB1 receptor is selected from the group consisting of an allosteric modulation, an orthosteric modulation, and a combination thereof.

According to some embodiments, the condition related to cannabinoid type 1 (CB1) receptor-activation is selected from the group consisting of pain, a sleep disorder, an appetite disorder, anxiety, depression, a memory disorder, a movement disorder, inflammation, abnormal excitatory neuronal activity, neurodegeneration, abnormal neurotransmitter release, stress, a cardiovascular function disorder, a motivation disorder, a mood disorder, a sedation disorder, a cognitive function disorder, abnormal muscle tension, cramps and combinations thereof.

According to a further aspect of some embodiments of the present invention there is provided a method for treating a condition related to Transient Receptor Potential Cation Channel, subfamily A, member 1 (TRPA1) receptor activation in a subject in need thereof, the method comprising modulating TRPA1 receptor activation by administering to the subject a composition comprising at least one terpene (such as one terpene, two terpenes, three terpenes, four terpenes or five terpenes) selected from the group consisting of linalool, menthol, eucalyptol, terpineol, citral and combinations thereof.

According to some embodiments, a total concentration of the at least one terpene in the composition is about 500 micromolar.

According to some embodiments, the method further comprises administering to the subject tetrahydrocannabinol (THC) at a total terpene to THC weight/weight ratio in the range between 0.05:1 and 1:1 (such as about 0.05:1, about 0.06:1, about 0.07:1, about 0.08:1, about 0.09:1, about 0.1:1, about 0.2:1, about 0.3:1. about 0.4:1, about 0.5:1, about 0.6:1, about 0.7:1, about 0.8:1, about 0.9:1 or about 1:1), wherein modulating TRPA1 receptor activation is via modulating an interaction of THC with TRPA1.

According to some embodiments, THC is administered to the subject at a dosage of from about 1 to about 100 mg (such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg) THC.

According to some embodiments, the at least one terpene is administered in a single composition together with the THC.

According to some embodiments, the at least one terpene is administered in a separate composition from the THC.

According to some embodiments, the at least one terpene is administered prior to administering of the THC.

According to some embodiments, the at least one terpene is administered subsequent to administering of the THC.

According to some embodiments, the at least one terpene is administered within 2 hours of administering of the THC.

According to some embodiments, the composition comprising at least one terpene is in a form selected from the group consisting of a vapor and an aerosol.

According to some embodiments, the THC is administered sublingually.

According to some embodiments, the composition comprising at least one terpene is administered in a liquid form.

According to some embodiments, modulating an interaction of the THC with the TRPA1 receptor is selected from the group consisting of an allosteric interaction, an orthosteric interaction, and a combination thereof.

According to some embodiments, the condition related to TRPA1 receptor activation is selected from the group consisting of pain, nociception, thermal sensation, thermal nociception, itch, burning sensation, irritation, airway disturbances, cough, inflammation, addiction, anxiety, depression, stress, abnormal cardiovascular function, abnormal muscle tension and combinations thereof.

According to a further aspect of some embodiments of the present invention there is provided a composition comprising at least one terpene (such as one terpene, two terpenes, three terpenes, four terpenes or five terpenes) selected from the group consisting of linalool, menthol, eucalyptol, terpineol, citral and combinations thereof for treating a condition related to Transient Receptor Potential Cation Channel, subfamily A, member 1 (TRPA1) receptor activation by modulating TRPA1 receptor activation.

According to some embodiments, a total concentration of the at least one terpene in the composition is about 500 micromolar.

According to some embodiments, the composition is for administering together with tetrahydrocannabinol (THC) at a total terpene to THC weight/weight ratio in the range between 0.05:1 and 1:1 (such as about 0.05:1, about 0.06:1, about 0.07:1, about 0.08:1, about 0.09:1, about 0.1:1, about 0.2:1, about 0.3:1, about 0.4:1, about 0.5:1, about 0.6:1, about 0.7:1, about 0.8:1, about 0.9:1 or about 1:1), wherein modulating TRPA1 receptor activation is via modulating an interaction of THC with TRPA1.

According to some embodiments, THC is for administering to a subject at a dosage of from about 1 to about 100 mg (such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg) THC.

According to some embodiments, the at least one terpene is for administering in a in a single composition together with the THC.

According to some embodiments, the at least one terpene is for administering in a separate composition from the THC.

According to some embodiments, the at least one terpene is for administering prior to administering of the THC.

According to some embodiments, the at least one terpene is for administering subsequent to administering of the THC.

According to some embodiments, the at least one terpene is for administering within 2 hours of administering of the THC.

According to some embodiments, the composition comprising at least one terpene is for administering in a form selected from the group consisting of a vapor and an aerosol.

According to some embodiments, the THC is for administering sublingually.

According to some embodiments, the composition comprising at least one terpene is for administering in a liquid form.

According to some embodiments, modulating an interaction of the THC with the TRPA1 receptor is selected from the group consisting of an allosteric interaction, an orthosteric interaction, and a combination thereof.

According to some embodiments, the condition related to TRPA1 receptor activation is selected from the group consisting of pain, nociception, thermal sensation, thermal nociception, itch, burning sensation, irritation, airway disturbances, cough, inflammation, addiction, anxiety, depression, stress, abnormal cardiovascular function, abnormal muscle tension and combinations thereof.

According to a further aspect of some embodiments of the present invention, there is provided a method for treating a condition related to Transient Receptor Potential Cation Channel, subfamily V, member 1 (TRPV1) receptor activation in a subject in need thereof, the method comprising modulating TRPV1 activation by administering to the subject a composition comprising at least one terpene selected from the group consisting of citral, caryophyllene and combinations thereof.

According to some embodiments, a total concentration of the at least one terpene in said composition is about 500 micromolar.

According to some embodiments, the method further comprises administering to the subject cannabidiol (CBD)) at a total terpene to CBD weight/weight ratio in the range between 0.05:1 and 1:1 (such as about 0.05:1, about 0.06:1, about 0.07:1, about 0.08:1, about 0.09:1, about 0.1:1, about 0.2:1, about 0.3:1, about 0.4:1, about 0.5:1, about 0.6:1, about 0.7:1, about 0.8:1, about 0.9:1 or about 1:1), wherein modulating TRPV1 receptor activation is via modulating an interaction of the CBD with the TRPV1 receptor.

According to some embodiments, the CBD is administered to the subject at a dosage of from about 1 to about 100 mg (such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg) CBD.

According to some embodiments, the at least one terpene is administered in a single composition together with the CBD.

According to some embodiments, the at least one terpene is administered in a separate composition from the CBD.

According to some embodiments, the at least one terpene is administered prior to administering of the CBD.

According to some embodiments, the at least one terpene is administered subsequent to administering of the CBD.

According to some embodiments, the at least one terpene is administered within 2 hours of administering of the THC.

According to some embodiments, the composition comprising the at least one terpene is administered in a form selected from the group consisting of a vapor and an aerosol.

According to some embodiments, the CBD is administered sublingually.

According to some embodiments, the composition comprising at least one terpene is administered in a liquid form.

According to some embodiments, modulating an interaction of the CBD with the TRPV1 receptor is selected from the group consisting of an allosteric modulation, an orthosteric modulation, and a combination thereof.

According to some embodiments, the condition related to TRPV1 receptor activation is selected from the group consisting of pain, nociception, thermal sensation, thermal nociception, itch, burning sensation, irritation, airway disturbances, cough, inflammation, addiction, anxiety, depression, stress, abnormal cardiovascular function, abnormal muscle tension and combinations thereof.

According to a further aspect of some embodiments of the present invention, there is provided a composition comprising at least one terpene selected from the group consisting of linalool, menthol, eucalyptol, terpineol, citral and combinations thereof for use in treating a condition related to Transient Receptor Potential Cation Channel, subfamily V, member 1 (TRPV1) receptor activation by modulating TRPV1 receptor activation.

According to some embodiments, a total concentration of the at least one terpene in the composition is about 500 micromolar.

According to some embodiments, the composition comprising the at least one terpene is for administering together with cannabidiol (CBD) at a total terpene to CBD weight/weight ratio in the range between 0.05:1 and 1:1 such as about 0.05:1, about 0.06:1, about 0.07:1, about 0.08:1, about 0.09:1, about 0.1:1, about 0.2:1, about 0.3:1, about 0.4:1, about 0.5:1, about 0.6:1, about 0.7:1, about 0.8:1, about 0.9:1 or about 1:1, wherein modulating the TRPV1 receptor is via modulating an interaction of the CBD with the TRPV1 receptor.

According to some embodiments, the CBD is for administering to a subject at a dosage of from about 1 to about 100 mg CBD.

According to some embodiments, the composition comprising the at least one terpene is for administering in a single composition together with the CBD.

According to some embodiments, the composition comprising the at least one terpene is for administering in a separate composition from the CBD.

According to some embodiments, the composition comprising the at least one terpene is for administering prior to administering of the CBD.

According to some embodiments, the composition comprising the at least one terpene is for administering subsequent to administering of the CBD.

According to some embodiments, the composition comprising the at least one terpene is for administering within 2 hours of administering of the CBD.

According to some embodiments, the composition comprising the at least one terpene is for administering in a form selected from the group consisting of a vapor and an aerosol.

According to some embodiments, the composition comprising the at least one terpene is for administering sublingually.

According to some embodiments, the composition comprising the at least one terpene is for administering in a liquid form.

According to some embodiments, modulating an interaction of CBD with the TRPV1 receptor is selected from the group consisting of an allosteric modulation, an orthosteric modulation, and a combination thereof.

According to some embodiments, the condition related to TRPV1 receptor activation is selected from the group consisting of pain, nociception, thermal sensation, thermal nociception, itch, burning sensation, irritation, airway disturbances, cough, inflammation, addiction, anxiety, depression, stress, abnormal cardiovascular function, abnormal muscle tension and combinations thereof.

### EXAMPLES

### Example 1

### Modulation of CB1 Receptor Activation by Terpenes

Methods and Materials

To test the possibility that the presence of various terpenes affects the activation of the CB1 receptor, the Inventors used Xenopus oocytes as a functional expression system. To this end, Xenopus oocytes were injected with cRNAs of proteins involved in the pathway leading to activation of K+ currents by CB1 receptor via βγ subunits of the G-proteins: The CB1 receptor, the two subunits of the GIRK channel (GIRK1 and GIRK2), and the Gαi3 subunit.

Preparation of cRNA and Oocytes:

cDNA plasmids of the two subunits (GIRK1 and GIRK2) of the G protein-coupled inwardly-rectifying potassium (GIRK) receptor, the CB1 receptor, and the α subunit of the G-protein (Gαi3) were linearized with the appropriate restriction enzymes. The linearized plasmids were transcribed in vitro using a standard procedure.

X. laevis oocytes were isolated and incubated in NDE96 solution composed of ND96 (in mM: 96 NaCl, 2 KCl, 1 CaCl2, 1 MgCl2, 5 Hepes, with pH adjusted to 7.5 with NaOH) with the addition of 2.5 mM Na+ pyruvate, 100 units/ml penicillin, and 100 μg/ml streptomycin. A day after their isolation, the oocytes were injected with the relevant cRNAs. cRNAs of CB1 receptor (2 ng) and GIRK1 and GIRK2 (200 pg each) were injected. In addition, cRNA of Gαi3 (1000 pg) was injected to decrease the basal GIRK current (IK) and to improve the relative activation by the agonist (17).

### Current Measurements:

Currents were measured 3-5 days after cRNA injection and were recorded using the standard two-electrode voltage clamp technique (Axoclamp 2B amplifier, Axon Instruments, Foster City, CA). Each oocyte was placed in the recording bath containing ND96 solution and was impaled with two electrodes pulled from 1.5-mm Clark capillaries (CEI, Pangboure, England). Both electrodes were filled with 3M KCl solution and the electrodes resistances were between 1-5 MΩ). The CB1-mediated GIRK currents were measured in a 24 mM K+ solution (in mM: 72 NaCl, 24 KCl, 1 CaCl2, 1 MgCl2, 5 Hepes, with pH adjusted to 7.5 with KOH). pCLAMP10 software (Axon Instruments) was used for data acquisition and analysis.

### Receptor Activation Measurements:

It was first verified that the compounds to be used in this study do not exert a receptor-independent effect on the GIRK channels. To do so, the effect of THC and a panel of various terpenes was measured on oocytes expressing the GIRK channel but not the CB1 receptor. None of these compounds showed an effect on the GIRK current, thus any current evoked in CB1 receptor-expressing oocytes may be attributed to the activation of the receptor.

Next, the dependence of the THC-induced K+ current (I_{THC}) on THC concentration (dose-response) was measured. The oocyte was voltage-clamped to-80 mV in a low K+ (2 mM K+) solution, ND96. Basal GIRK current (I_{K}) was developed upon replacement of the ND96 by the 24 mM K+ solution. This current represents the basal activation of GIRK channel by endogenous βγ subunits present in the oocytes. Then, five concentrations of THC were applied sequentially, giving rise to ITHC. This current was terminated upon washout of THC. Employing this basic experimental protocol, a full dose-response curve was constructed. To be able to compare between oocytes, I_{THC} at any particular THC concentration was normalized to I_{THC} obtained at a 10 μM, defined as the reference response.

Next, the effect of terpenes on CB1 activation was assessed.

### Results

Administration of 10 μM terpene produced CB1 receptor activation at a magnitude of 23-48% of the activation obtained by THC at the CB1 receptor.

Response amplitudes were normalized to the response obtained with 10 μM THC.

TABLE 2

| Terpene | Response to 10 μM terpene |
|---|---|
| α-pinene | 0.29 ± 0.03 |
| β-pinene | 0.29 ± 0.06 |
| eucalyptol | 0.41 ± 0.04 |
| geraniol | 0.31 ± 0.08 |
| limonene | 0.23 ± 0.02 |
| myrcene | 0.26 ± 0.07 |
| ocimene | 0.36 ± 0.05 |
| terpinolene | 0.45 ± 0.02 |
| terpineol | 0.48 ± 0.05 |

### Example 2

### Modulation of CBI Receptor Activation by Terpenes in Combination with THC

Xenopus oocytes were used as a functional expression system, as described in Example 1.

Terpenes were administered together with tetrahydrocannabinol (THC) at a terpene to THC weight/weight ratio in the range between 0.05:1 and 1:1.

Administration of THC together with a terpene at a terpene to THC weight/weight ratio of 0.1:1 produced CB1 receptor activation significantly higher than the receptor activation obtained using THC alone at the same concentration. Augmented activation was given as fold change. Fold change in potency is given as the ratio between the THC concentration required to evoke 50% of the maximal response in the presence of terpene and the THC concentration that evoked the same response in the absence of terpene.

TABLE 3

| | Fold change of THC potency by terpene, |
|---|---|
| Terpene | at terpene:THC ratio of 1:10 |
| borneol | 4.469 |
| geraniol | 1.576 |
| limonene | 3.369 |
| linalool | 1.553 |
| ocimene | 2.639 |
| sabinene | 1.685 |
| terpineol | 2.704 |

### Example 3

### Modulation of TRPA1 Receptor Activation by Terpenes

### Methods and Materials

### Cell Culturing and Transfection

rTRPV1 or hTRPA1 were stably expressed in TREx 293 cells following the manufacturer's protocol (Invitrogen, MA, USA). Briefly, TREx host cell line (Invitrogen, MA, USA) was transfected with pcDNA4/TO containing the gene of interest using Mirus LT1 transfection reagent (Mirus Bio, WI, USA) with Opti-MEM I Reduced Serum Medium (Invitrogen, MA, USA). Successful recombination and maintenance were confirmed through zeocin (500 μg/ml; Invivogen, Toulouse, France) selection to establish a stably-transfected cell line. Cells were grown in DMEM (Sigma-Aldrich, MO, USA) supplemented with 10% FBS, 1% penicillin-streptomycin, 2 mM L-alanine L-glutamine, and 25 mM HEPES (pH 7.3) supplemented with 5 μg/ml blasticidin at 37° C. and 5% CO2. Cells were passaged twice per week. Three hours before analysis, cells were treated with doxycycline (0.2-1 μg/ml) to induce transgene expression.

### Live-Cell Calcium Imaging

Flip-in TREX293T cells were spotted at PDL (0.2 mg/ml)-coated imaging chambers (μ-slide, 8-well; Ibidi, Martinsried, Germany) and incubated for 1.5-2 h in a humidified incubator at 37° C. and 5% CO2. Cells were loaded with 3 μg/ml Fura-2 AM (Thermo Fisher Scientific) dissolved in Ringer solution. The imaging chamber was placed onto the inverted microscope (IX70; Olympus, Tokyo, Japan). Cells were illuminated with a 175W xenon arc lamp, and excitation wavelengths (340/380 nm) were selected by a Lambda DG-4 monochromatic wavelength changer (Sutter Instrument, Novato, CA, USA). Intracellular Ca2+ concentration was measured by digital video microfluorometry illuminated interline CCD camera (Exi Blue; QImaging, Surrey, BC, Canada) using MetaFluor fluorescence ratio imaging software (Molecular Devices). Dual images (340 and 380 nm excitation, 510 nm emission) were collected, and pseudocolor ratio-metric images were monitored every 4 s during the experiment. Data analysis was performed with MetaFluor fluorescence ratio imaging software. Data are presented as a normalized intensity of baseline fluorescence. All experiments were carried out at room temperature.

### Results TRPA1

Results are summarized in Table 2. Data for THC (leftmost column) and for THC with a 1/10 concentration of a terpene are presented. Response amplitudes are normalized to the maximal activation obtained by administration of Allyl Isothiocyanate (AITC). Numbers in parentheses represent p values for the comparison of the TRPA1 activation for THC and for THC plus a 1/10 concentration terpene.

TABLE 4

| | THC | Linalool | Menthol | Eucalyptol | Terpineol | Citral |
|---|---|---|---|---|---|---|
| 8 μM | 0.35 | 0.44 | 0.44 | 0.48 | | |
| THC | | (0.02) | (0.02) | (0.008) | | |
| 30 μM | 0.8 | 0.93 | | 0.93 | 0.97 | 0.96 |
| THC | | (<0.0001) | | (<0.0001) | (<0.0001) | (<0.0001) |

1-128. (canceled)

129. A method for treating a condition related to cannabinoid type 1 (CB1) receptor activation selected from the group consisting of pain, a sleep disorder, an appetite disorder, anxiety, depression, a memory disorder, a movement disorder, inflammation, abnormal excitatory neuronal activity, neurodegeneration, abnormal neurotransmitter release, stress, a cardiovascular function disorder, a motivation disorder, a mood disorder, a sedation disorder, a cognitive function disorder, abnormal muscle tension, cramps and combinations thereof or a symptom thereof in a subject in need thereof, the method comprising modulating CB1-receptor activation by administering to the subject a composition comprising at least one terpene selected from the group consisting of alpha pinene, beta pinene, limonene, terpineol, geraniol, myrcene, ocimene, terpinolene, eucalyptol, linalool, borneol, sabinene and combinations thereof.

130. The method of claim 129, further comprising administering to the subject tetrahydrocannabinol (THC) at a total terpene to THC weight/weight ratio in a range between 0.05:1 and 1:1, wherein said modulating CB1-receptor activation is via modulating an interaction of said THC with said CB1 receptor.

131. The method of claim 130, wherein said THC is administered to the subject at a dosage of from about 1 to about 100 mg THC.

132. The method of claim 130, wherein said at least one terpene is selected from the group consisting of linalool, limonene, geraniol, ocimene, borneol, terpineol, sabinene, beta pinene and combinations thereof.

133. The method of claim 129, wherein said at least one terpene comprises at least three terpenes selected from the group consisting of alpha pinene, myrcene, limonene, linalool, caryophyllene, humulene, nerolidol and, wherein said alpha pinene, when present, is at a concentration of from about 5 to about 20 wt % of the total terpene content; wherein said myrcene, when present, is at a concentration of from about 5 to about 25 wt % of the total terpene content; wherein said limonene, when present, is at a concentration of from about 1 to about 15 wt % of the total terpene content; wherein said linalool, when present is at a concentration of from about 3 to about 20 wt % of the total terpene content; wherein said caryophyllene, when present, is at a concentration of from about 15 to about 35 wt % of the total terpene content; wherein said humulene, when present, is at a concentration of from about 1 to about 15% of the total terpene content; and wherein said nerolidol, when present, is at a concentration of from about 5 to about 25 wt % of the total terpene content.

134. The method of claim 129, wherein said composition comprises at least three terpenes selected from the group consisting of alpha pinene, limonene, terpinolene, caryophyllene and bisabolol, wherein said alpha pinene, when present, is at a concentration of from about 15 to about 35 wt % of the total terpene content; wherein said limonene, when present, is at a concentration of from about 10 to about 30 wt % of the total terpene content; wherein said terpinolene, when present, is at a concentration of from about 5 to about 25 wt % of the total terpene content; wherein said caryophyllene, when present, is at a concentration of from about 15 to about 35 wt % of the total terpene content; and wherein said bisabolol, when present, is at a concentration of from about 3 to about 20 wt % of the total terpene content.

135. The method of claim 129, wherein said composition comprises at least three terpenes selected from the group consisting of beta pinene, myrcene, ocimene, linalool, terpineol, borneol, caryophyllene, and nerolidol, wherein said beta pinene, when present, is at a concentration of from about 5 to about 25 wt % of the total terpene content, wherein said myrcene, when present, is at a concentration of from about 3 to about 25 wt % of the total terpene content; wherein said ocimene, when present, is at a concentration of from about 3 to about 20 wt % of the total terpene content;
wherein said linalool, when present, is at a concentration of from about 5 to about 25 wt % of the total terpene content; wherein said terpineol, when present, is at a concentration of from about 5 to about 25 wt % of the total terpene content; wherein said borneol, when present, is at a concentration of from about 5 to about 25 wt % of the total terpene content; wherein said caryophyllene, when present, is at a concentration of from about 10 to about 30 wt % of the total terpene content; and wherein said nerolidol, when present, is at a concentration of from about 5 to about 25 wt % of the total terpene content.

136. The method of claim 129, wherein said composition comprises at least three terpenes selected from the group consisting of alpha pinene, sabinene, limonene, terpinolene, eucalyptol, borneol and caryophyllene, wherein said alpha pinene, when present, is at a concentration of from about 5 to about 25 wt % of the total terpene content; wherein said sabinene, when present, is at a concentration of from about 5 to about 25 wt % of the total terpene content; wherein said limonene, when present, is at a concentration of from about 5 to about 25 wt % of the total terpene content;
wherein said terpinolene, when present, is at a concentration of from about 1 to about 15 wt % of the total terpene content; wherein said eucalyptol, when present, is at a concentration of from about 5 to about 25 wt % of the total terpene content; wherein said borneol, when present, is at a concentration of from about 5 to about 25 wt % of the total terpene content; and wherein said caryophyllene, when present is at a concentration of from about 5 to about 30 wt % of the total terpene content.

137. The method of claim 129, wherein said composition comprises at least three terpenes selected from the group consisting of alpha pinene, beta pinene, eucalyptol, linalool, terpineol, borneol, caryophyllene and nerolidol, wherein said alpha pinene, when present, is at a concentration of from about 5 to about 25 wt % of the total terpene content; wherein said beta pinene, when present, is at a concentration of from about 1 to about 15 wt % of the total terpene content; wherein said eucalyptol, when present is at a concentration of from about 3 to about 25 wt % of the total terpene content; wherein said linalool, when present, is at a concentration of from about 3 to about 25 wt % of the total terpene content; wherein said terpineol, when present, is at a concentration of from about 3 to about 25 wt % of the total terpene content; wherein said borneol, when present, is at a concentration of from about 3 to about 25 wt % of the total terpene content; wherein said caryophyllene, when present, is at a concentration of from about 5 to about 25 wt % of the total terpene content; and wherein said nerolidol, when present is at a concentration of from about 3 to about 25 wt % of the total terpene content.

138. The method of claim 129, wherein said composition comprises at least three terpenes selected from the group consisting of alpha pinene, myrcene, caryophyllene and nerolidol, wherein said alpha pinene, when present, is at a concentration of from about 5 to about 25 wt % of the total terpene content; wherein said myrcene, when present, is at a concentration of from about 15 to about 35 wt % of the total terpene content; wherein said caryophyllene, when present, is at a concentration of from about 15 to about 35 wt % of the total terpene content; and wherein said nerolidol, when present is at a concentration of from about 15 to about 35 wt % of the total terpene content.

139. The method of claim 129, wherein said at least one terpene comprises at least two terpenes selected from the group consisting of limonene, alpha pinene, borneol, bisabolol and myrcene.

140. The method of claim 129, wherein said at least one terpene is selected from the group consisting of alpha pinene, beta pinene, limonene, terpineol, geraniol, myrcene, ocimene, terpinolene, eucalyptol and combinations thereof.

141. The method of claim 129, wherein said at least one terpene is selected from the group consisting of alpha-pinene, beta-pinene, ocimene, limonene, linalool, geraniol, terpinolene, valencene, and selinadiene and combinations thereof.

142. The method of claim 129, wherein said at least one terpene comprises at least three terpenes selected from the group consisting of alpha-pinene, beta-pinene, limonene, terpinolene, geraniol, caryophyllene, bisabolol and combinations thereof.

143. The method of claim 130, wherein said alpha pinene, when present, is at a concentration of from about 5 to about 25 wt % of the total terpene content; wherein said beta pinene, when present, is at a concentration of from about 1 to about 15 wt % of the total terpene content; wherein said limonene, when present, is at a concentration of from about 5 to about 25 wt % of the total terpene content; wherein said terpinolene, when present, is at a concentration of from about 5 to about 25 wt % of the total terpene content; wherein said geraniol, when present is at a concentration of from about 5 to about 25 wt % of the total terpene content; wherein said caryophyllene, when present, is at a concentration of from about 10 to about 30 wt % of the total terpene content; and wherein said bisabolol, when present, is at a concentration of from about 5 to about 25 wt % of the total terpene content.

144. The method of claim 130, wherein said composition comprising said at least one terpene is administered in a separate composition from said THC.

145. A method for treating a condition related to Transient Receptor Potential Cation Channel, subfamily A, member 1 (TRPA1) receptor activation selected from the group consisting of pain, nociception, thermal sensation, thermal nociception, itch, burning sensation, irritation, airway disturbances, cough, inflammation, addiction, anxiety, depression, stress, abnormal cardiovascular function, abnormal muscle tension and combinations thereof in a subject in need thereof, the method comprising modulating TRPA1 receptor activation by administering to the subject a composition comprising at least one terpene selected from the group consisting of linalool, menthol, eucalyptol, terpineol, citral and combinations thereof.

146. The method of claim 145, further comprising administering to the subject tetrahydrocannabinol (THC) at a total terpene to THC weight/weight ratio in the range between 0.05:1 and 1:1, wherein said modulating TRPA1 receptor activation is via modulating an interaction of said THC with said TRPA1.

147. A method for treating a condition related to Transient Receptor Potential Cation Channel, subfamily V, member 1 (TRPV1) receptor activation selected from the group consisting of pain, nociception, thermal sensation, thermal nociception, itch, burning sensation, irritation, airway disturbances, cough, inflammation, addiction, anxiety, depression, stress, abnormal cardiovascular function, abnormal muscle tension in a subject in need thereof, the method comprising modulating TRPV1 activation by administering to the subject a composition comprising at least one terpene selected from the group consisting of citral, caryophyllene and combinations thereof.

148. The method of claim 147, further comprising administering to the subject cannabidiol (CBD) at a total terpene to CBD weight/weight ratio in the range between 0.05:1 and 1:1, wherein said modulating TRPV1 receptor activation is via modulating an interaction of said CBD with said TRPV1 receptor, wherein said CBD is administered to the subject at a dosage of from about 1 to about 100 mg CBD.
